(19) United States
(12) Patent Application Publication
(10) Pub. No.: US 2008/0045990 A1
(43) Pub. Date: Feb. 21, 2008

(54) Combination medical bandage scissors

(75) Inventor: Sharon Jane Zelinko, Winter Haven, FL (US)

Correspondence Address: SHARON JANE ZELINKO, 121 SAND BURG LANE, WINTER HAVEN, FL, 33884, US

(73) Assignee: Susan Lynn Valerio, Lock Haven, PA (US)

(21) Appl. No.: 11/503,882

(22) Filed: Aug. 15, 2006

(51) Int. Cl. A61B 17/32

(52) U.S. Cl. 606/174

(57) ABSTRACT

Improved angular shear or scissors comprising two mated scissor elements, each of which comprises a blade section, a shank section and a handle section, said elements being pivotally united at a position between blade and shank whereby cutting action can occur when handle sections are manipulated. Other standard scissors feature is a blunt tip that prevents piercing as a safety measure. Medical procedures require a greater range of mechanical assistance than these scissors can provide—mostly related to gripping action.

The object of the present invention is to provide gripping ability to significantly increase the usefulness of these scissors by practitioners. The grip provided by the utility created by this modification can be used to grip-in-order-to-twist (rotating motion), grip to hold (stationary), or grip to insert or extract (straight motions). The grooves must be fine in order to prevent cutting manipulated items, maintaining safety of original unmodified scissors.

### INTRODUCTION

Gripsors brand combination medical bandage scissors incorporate an important modification of fine grooves between the handles of medical bandage scissors for left- or right-handed users in three sizes for use in a wide range of standard and specialized medical practice including neo natal use (small size), emergency services (larger size scissors for cutting off clothing), and field military applications, which also involves larger-than-standard-size medical bandage scissors.

### BACKGROUND

Need was the inspiration for my idea. Many times I am unable to twist the IV tubing off when it needs to be changed because the person before me turned it too tightly. I always carry a hemostat with me to loosen it. Nurses always carry bandage scissors with them. For years I thought how convenient it would be to have one tool that served both purposes. In my current job other nurses consistently come to me to borrow my hemostat because they have the same problem with the IV tubing and they know that I carry a hemostat for that purpose. I am convinced that this invention has incredible market potential due to my own professional practice as a registered nurse. Over the 15 years since I thought of this idea, I expected that someone would invent a tool such as this and market it. When this did not happen, it occurred to me that I might be the person to do it. I fully expect that a well designed pair of scissors capable of twisting IV tubing or G(astrostomy) tubing (as well as other difficult-to-manipulate items) as a feature could completely replace the bandage scissors currently on the market. These scissors could also serve veterinary applications providing medical care to animals. The utility involves enabling the user to grip in order to twist, insert, or extract. The applications are without limit. Nurses have reported they use these scissors to open nail polish bottles at home. While we are marketing them to medical/veterinary service providers, in reality their usefulness extends beyond this sphere.

### BRIEF SUMMARY OF THE INVENTION

Gripsors brand combination medical bandage scissors are stainless steel construction. The scissors themselves are purchased in finished form for left- or right-handed users in three sizes:
SMALL—Neo natal/dental applications or for small animals in veterinary applications (approx. 3.5-inch overall length)
STANDARD—General medical applications or for larger animals in veterinary applications (approx. 5.5-inch overall length)
LARGE—Emergency/field military applications where scissors must be used to cut off clothing, or for large animals in veterinary applications (approx. 7.5-inch overall length)

The modification requested for patent is a single modification and does not vary except in the location of the modification in the shaft, the purposes accomplished in the utility provided are also the same for the most part (tiny applications in neo natal units, tiny scissors; larger applications elsewhere, larger scissors—all uses for medical applications requiring gripping for twisting or inserting or extracting). Therefore, the inventor believes the variations as stated clearly demonstrate this unique and similar feature and can be covered under a single patent.

### DESCRIPTION OF DRAWING

The technical drawing provided with this application uses standard size scissors for right-handed users. The modification and style of the scissors does not vary so drawings to illustrate the modification are on a single page.

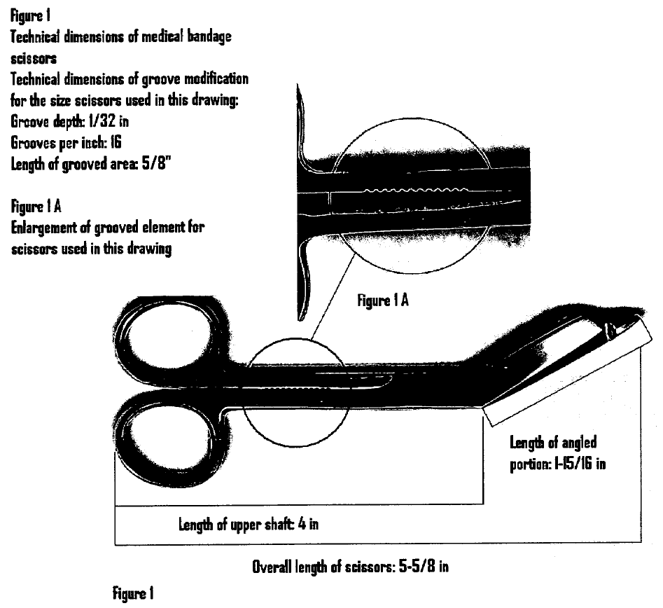

1) The drawing is an actual photograph of the scissors (FIG. 1) with a close up inset photo (FIG. 1A) highlighting the grooved area of the scissors.

2) The drawing identifies the construction of these scissors as stainless steel.

3) The drawing provides overall scissors measurement for the standard size used in the technical drawing (FIG. 1).

4) The drawing specifies the length of the shaft and angled portion of the scissors in separate measurements (FIG. 1).

5) The drawing states the length of the grooved area and states the number of teeth per inch present in the finished scissors.

6) The drawing states the depth of the grooves in the finished scissors.

The scissors in this drawing were scanned into a computer file. The full page drawing submitted with this non-provisional application for patent was obtained from an electronic file by using the Windows Print Wizard and Fax Viewer and requesting a full page printing.

The modification for which a patent is requested is fine grooves between the handles. The grooving is accomplished by the following method:

### DESCRIPTION OF TECHNICAL PROCESS FOR ACCOMPLISHING MODIFICATION

Using stainless steel medical bandage scissors as shown in the technical drawing, The gripping teeth are located 1.580 from the outer end of the hoop to the center of the first “V”. The length of the teeth measures 0.660 in length starting from the center of the first “V” groove to the center of the last “V” groove, there are a total of 12 “V” grooves. There are 11 teeth each side of the tooth is machined at a 45 degree angle from the center of the “V” groove. The depth of the “V” groove measures 0.020 from the top of the tooth to the bottom of the grove. Each tooth is spaced at 0.060 from the center point of each tooth. The point of the tooth is designed to have 0.015 flat at the top of the tooth (the tooth doesn't come to a sharp point). The bottom of the “V” groove doesn't come to a sharp point; this is designed to have a 0.005 radius at the bottom.

The teeth are cut into the inside of the handle. One set of teeth on each handle of each scissor. This operation is called milling and the machine used is called a horizontal milling machine. The scissor handle is fixtured and held in an air vise which is mounted to the table of the horizontal milling machine. Once the operator has the handle securely clamped in the vise, a lever is tripped and the table moves the scissor underneath a circular cutter. This cutter is special ground to match the shape of the teeth as described above. Once the cutter passes over the handle and has cut the shaped teeth into the handle the table retracts back to a safe area for the operator to reload. The operator removes the scissor turns it 180 degrees, reloads the other half of the scissor handle and repeats the operation. There are two cycles required to complete one pair of scissors.

Because oil is use to lubricate the cutters, a cleaning operation is performed to remove the oil. The scissors are then bulk packed for delivery.

This description is good for both the 5.5 scissor and the 7.5 scissor.

These modifications are slightly finer and the grooved area smaller for the 3.5 scissor used in neo-natal applications.

### DETAILED DESCRIPTION

Medical bandage scissors are carried in the pocket of medical personnel as an essential/standard item. The gripping action provided by the grooves is needed frequently in the course of normal medical procedures where medical bandage scissors are carried by medical personnel. Combining both uses in one item provides significant benefits in that the ability to grip is always readily available for use, which is needed much more frequently than cutting in the course of medical care.

The inventor is a practicing nurse who knows first hand the benefits of the proposed modification. Placing the grooves in the handle of the scissors allows for items of different diameters to benefit from the ability to grip—from very small to approximately one-inch in diameter in the standard scissor application provided with scissors dimensions provided in the technical drawing in this provisional application for patent (standard size). This means the scissors can be used in a wide range of applications providing additional benefits to the user. The grip provided by the utility created by this modification can be used to grip-in-order-to-twist (rotating motion), grip to hold (stationary), grip to insert or extract (straight motions). The applications are without limit. Nurses have reported they also use these scissors to open nail polish bottles at home. While these scissors are being marketed to medical service providers, in reality their usefulness extends beyond the medical sphere.

Medical bandage scissors are designed with a blunt tip to prevent inadvertent piercing of skin or through the pocket of the clothing item being worn by medical personnel. The blunt tips also prevent puncture of other items in the vicinity where the gripping action is needed.

The grooves of the modification must be fine and blunted so they will not cut the item being held or manipulated. These items include IV tubing and connections, G tubing and connections, oxygen tubing and connections, vials, items difficult to insert or extract. The grooves in the example submitted in this provisional application for patent are indicated on the technical drawing and described in the technical description. The grooving may vary slightly in smaller applications, such as in the length of the grooved area in the shaft between the handles, but the important feature is that the grooves are shallow and fine preventing them from cutting into tubing, and sufficiently ridged to permit effective gripping action.

Potential cost savings have been reported to marketers in the early distribution of these scissors: Some nurses have reported that on occasion when they needed to open an IV and were unable to do so, an entire set of medical instruments have been opened and a gripping tool used to open an IV, then the entire packaged set of instruments was discarded and charged to the patient because it was essential to open the IV tubing and there was no other way available to do it. The inventor believes insurance companies and hospitals will recognize this claim and eventually require or sponsor the use of these scissors to avoid such a risk, both in risk to the patient when critical assistance is not readily available and in avoiding potential waste of medical supplies. See Appendix B.

No comparable item exists on the market. The inventor waited nearly 15 years for such a utility to be developed for medical use before taking steps to produce these modified scissors, which have been enthusiastically received and perceived by medical personnel as valuable, useful, and unique, and, therefore, worthy of pursuing a patent. Purchasers have been specifically asked if they feel this utility would be patentable. Without exception they see this utility as new and unique, not available anywhere else and, in their view, absolutely worthy of patent. See Appendices A and B.

The modification and utility accomplished in the combination medical bandage scissors for which this patent is sought are significant. They are not obvious due to the fact that so many years have passed before this modification was considered. The inventor considered this modification for 15 years before considering the possibility of creating these scissors even though practicing medicine and needing the utility these scissors provide on a constant basis.

The modification significantly adds to the utility of the unmodified medical bandage scissors used in this process.

### DRAWINGS

One page technical drawing with notes and measurements follows this page.

I claim:

1. A modification of fine grooves in the handle of said scissors allowing for items of different diameters to benefit from the ability to grip. The grip provided by the utility created by this modification can be used to grip-in-order-to-twist (rotating motion), or grip to hold (stationary), insert or extract (straight motions). Control over the utility is governed by the user in that the amount of pressure (intense to very gentle) needed to accomplish the desired manipulation can be determined by the user depending upon the application and the size of the item that requires manipulation.
